(19) United States
(12) Patent Application Publication
(10) Pub. No.: US 2004/0015066 A1
(43) Pub. Date: Jan. 22, 2004

(54) Fetal scalp electrode

(76) Inventor: Karl G. Rosen, Kungalv (SE)

Correspondence Address: McCormick Paulding & Huber, CityPlace II, 185 Asylum Street, Hartford, CT, 06103-3402, US

(21) Appl. No.: 10/380,556

(22) PCT Filed: Sep. 13, 2001
(86) PCT No.: PCT/GB01/04111

(30) Foreign Application Priority Data
Sep. 13, 2000 (GB) 0022484.0

(51) Int. Cl. A61B 5/04

(52) U.S. Cl. 600/376

(57) ABSTRACT

A fetal scalp electrode is provided having a spiral tip 5 mounted on the distal end of a dielectric hub 4 and a reference electrode 9 mounted on the proximal end thereof. Conventional apparatus including drive tube (3) and guide tube (2) are used to attach the spiral tip to the fetal scalp in a cork screw fashion. These components are removed leaving electrode wires (8) connected to spiral tip (5) and reference electrode (9) respectively extending from the fetus for connection to suitable monitoring equipment. The spiral tip (5) of the electrode is formed of stainless steel pype 905L which is essentially non-magnetic and non-magnetisable. This has been found to overcome a significant problem of prior art electrodes when used for detecting S-T wave intervals in particular where magnetic properties of the stainless steel typically used can lead to losses of critical portions of the signal. The electrode is particularly suitable for monitoring of the S-T interval in which it is used in a unipolar configuration, i.e. the reference electrode (9) is not used, but instead a remote electrode is attached to the maternal thigh. The wire connected to reference electrode (9) may, however, be simultaneously used for monitoring fetal heart rate.

The present invention relates to fetal scalp electrodes and in particular such electrodes which are suitable for use in producing an electro cardiogram (ECG) signal during delivery of the fetus.

Fetal surveillance during labour is standard clinical practice. Its purpose is to identify abnormal events and in particular fetal oxygen deficiency. At its simplest the fetal heart rate is monitored by determining the interval between successive R-wave peaks in the ECG signal. These peaks are by far the most pronounced portion of a normal ECG signal.

The conventional ECG lead configuration is the so-called bipolar configuration in which both electrodes are provided close to each other on the presenting part of the fetal body, i.e. the head or buttock. This is typically achieved by means of a specially designed fetal scalp electrode assembly which provides signals to two leads. The first is provided by a short pointed spiral electrode which is used to pierce the skin of the scalp and is then twisted to secure it into position in a corkscrew fashion. Needless to say, the electrode is carefully designed to ensure that the spiral cannot be pushed too far into the scalp. Sometimes a twin spiral arrangement is provided. The second contact is provided by an external portion of the electrode assembly which is insulated from the first and is arranged to make contact with the amniotic fluid. The amniotic fluid is in fact isolated from the spiral electrode when the latter is fully engaged by the body of the apparatus. Leads connected to the electrode assembly then transmit the ECG signal to suitable monitoring equipment.

This arrangement is widely used and has been found to be highly satisfactory for detecting fetal heart rate which provides valuable information to medical personnel. However, since the introduction of such fetal monitoring in the 1960s, it has been evident that electronic fetal monitoring by fetal heart rate analysis alone does not provide all of the information required for an optimum identification of a fetus suffering from lack of oxygen. Consequently, work has been ongoing to clarify what fetal signals, apart from the R-R interval, could be made use of to provide useful additional information. It was found in particular that the S-T interval and T-wave amplitude were of particular interest (see Rosén, K. G.: Fetal ECG wave form analysis in labour. Fetal monitoring. physiology and techniques of anti-natal and intrapartum assessment. ad. AD. Spencer J A D, Castle House Publications, pages 184-187, 1989).

These portions of the ECG signal are, however, more difficult to detect than the R-wave peaks since they have a much lower amplitude and can be difficult to distinguish from noise. For example, the amplitude changes of the S-T interval that may be detected are in the region of only 10 μV. Furthermore, since their main vectorial distribution is along the longitudinal axis of the fetus they cannot effectively be detected by the bipolar lead configuration discussed above. Consequently, the so-called unipolar fetal ECG lead configuration has been used (see Lindecrantz K, Lilja H, Widmark C, Rosén, K. G.: the fetal ECG during labour. A suggested standard. J. Biomed. Eng. 1998; 10: pages 351-353). In this configuration the spiral scalp electrode, as discussed above, is placed under the skin of the presenting fetal part, but instead of the second electrode being in contact with the amniotic fluid, an entirely separate electrode is used which is connected well away from the fetus, for example on the maternal thigh.

Although the unipolar fetal ECG electrode configuration enables the T wave vector in particular to be identified, a signal noise problem is generated at the same time. This is because the maternal skin electrode is sensitive to maternal movements causing both low frequency and high frequency noise. This noise is sufficient to make it difficult to derive useful information from the S-T waveform. It is therefore necessary to filter the signal. This has been done using analog filtering techniques and more recently by the digital filtering technique discussed in the inventor's earlier patent application published as GB 2342449A. Clinical trials have demonstrated that this enables highly reliable determination of fetal distress and can greatly reduce the number of unnecessary interventions.

However, the inventor found that when using this system, on an apparently random basis, occasionally a useful signal could not immediately be obtained. It was found that this problem could be overcome by the step of replacing the scalp electrode and so these electrodes were investigated. The apparently defective electrodes were tested for conductivity but no problem was found and furthermore, the electrodes were found to perform flawlessly when used to detect the R-R interval in the conventional manner. Thus, the inventors found that unexpectedly, and apparently randomly, a proportion of seemingly perfect electrodes tended to transmit a signal, but to distort it to such a degree that the low amplitude portions of the signal were useless.

The present invention addresses this unexpected problem which is solved according to one aspect by providing a fetal scalp electrode assembly having an electrode formed of a conductive material wherein the material is of a type which is substantially non-magnetic and furthermore does not become significantly magnetic after being cold worked.

The invention is thus based upon the insight that the undesirable behaviour of certain prior art electrode assemblies is caused by the magnetic behaviour of the stainless steel from which the electrode is formed. This depends upon the internal structure of the steel, which, as is well known, may contain structures known as austenite, ferrite and martensite. Surgical instruments are typically formed using martensite-containing stainless steels. These are well known to have magnetic properties, but even essentially austenitic grades of steel such as the very common grade 304 (EN 1.4301) from which scalp electrodes are conventionally manufactured can become significantly attracted to a magnet after being cold worked. The random nature of the problem occurring in the prior art may therefore be accounted for by random changes in the structure of the steel, and/or the presence of external magnetic fields as it is drawn and then formed into the spiral. The precise mechanism is not, however, fully understood, but neither is such an understanding essential to the efficacy of the invention. It has, however, been demonstrated that magnetic properties of the steel of the “problem” electrodes are to blame because such electrodes work perfectly for a while after being heated to the Curie temperature and then cooled. This produces an improvement of 300% in signal amplitude. Furthermore, non-magnetisable electrodes according to the invention which are otherwise identical to those of the prior art do not exhibit this problem.

It seems that the magnetic properties of the “problem” electrodes distort the weak signal which is being detected by reducing its amplitude in a non-linear fashion. In effect the electrode filters out certain frequencies of signal in an undesirable and unpredictable way.

It will be appreciated that stainless steel is generally the most suitable material for fetal scalp electrodes because it provides mechanical strength, freedom from corrosion and can be worked into the appropriate shape. It is therefore preferred that the fetal scalp electrode is formed from a grade stainless steel having the properties referred to above. Of course, other materials may also be used in the electrode and indeed it is preferred that an outer body of dielectric material be provided to isolate the spiral from the amniotic fluid and also that means be provided for allowing convenient connection between the spiral and the lead therefrom (which is typically formed of twisted copper). Indeed, the structure of the electrode (as opposed to the material from which the spiral is formed) may be entirely conventional.

When specifying grades of stainless steel it is common practice for the magnetic properties thereof to be described in terms of relative permeability (μᵣ). This should preferably be as low as possible, consistent with the other requirements of the stainless steel, such as strength and corrosion resistance. Thus, μᵣ of the material should be less than 5, preferably less than 3, more preferably less than 2 and ideally less than 1.2. Indeed, certain steel manufacturers define their steels as non-magnetic or non-magnetisable when μᵣ is in the range of 1.05-1.2 and it is particularly preferred that material having relative permeabilities within this range be used in the invention. It is to be observed that the material must be selected such that μᵣ is maintained within the desired range when cold worked and so materials such as stainless steel type 304 are not suitable.

It will be appreciated that austenitic grades of stainless steel are likely to be most appropriate, and ideally fully austenitic steels are used. Indeed, viewed from a further aspect the invention may be regarded as the provision of a fetal scalp electrode formed of fully austenitic stainless steel.

An example of such a steel which is particularly preferred for the present invention is grade 904L (otherwise known as EN 1.4539 and ASTM N08904) which as well as being non-magnetic is highly resistant to corrosion.

As previously discussed, the electrode may be otherwise conventional in form, but it is however preferred that only a single spiral be used as double spiral electrodes have been found to introduce a degree of signal noise.

Furthermore, although the electrode is suitable for, and indeed should be advantageous in conventional fetal heart rate monitoring, it is primarily intended for use in more advanced fetal monitoring systems, in particular those discussed above in which the S-T interval is studied. Consequently, the electrode may conveniently be provided in combination with just a single lead such that it may be used in the unipolar configuration discussed previously. Furthermore, in order to reduce costs and simplify the design it is preferably provided with only a single, spiral, electrode i.e. no provision is made for electrical connection to the amniotic fluid. Thus, the electrode of the invention is preferably used in the “unipolar” configuration, i.e. in combination with a separate electrode suitable for connection to, for example, the maternal thigh.

The invention also extends to a method of manufacturing a fetal scalp electrode comprising the steps of selecting a grade of material which is not magnetic even after cold working and forming a fetal scalp electrode therefrom.

The method preferably further comprises forming an electrode assembly according to one or more of the preferred features as previously set forth.

Viewed from a yet further aspect the invention provides a method of fetal monitoring comprising the use of an electrode of the invention as previously described.

Certain embodiments of the invention will now be described with reference to the accompanying drawing:

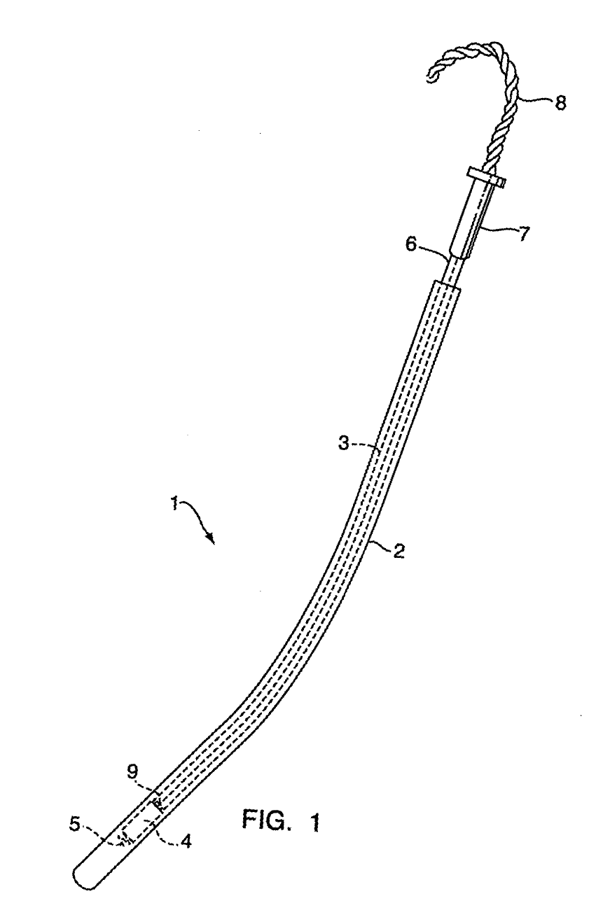

FIG. 1 is a perspective view of an electrode according to the invention.

FIG. 1 illustrates a fetal scalp electrode assembly 1 according to the invention including those components used to manipulate the electrode into position. Thus, the assembly 1 includes components which enable the electrodes to be applied to the fetal scalp whilst still in the uterus.

A guide tube 2 formed of fairly rigid plastic material surrounds drive tube 3 and hub 4 on which spiral tip 5 is mounted at the distal end. The proximal end 6 of the drive tube 7 extends from the guide tube 2. At this end is mounted drive tube grip 7 from which the electrode wires 8 extend for connection to monitoring apparatus. The electrode wires pass through the drive tube grip and through the guide tube where they are connected to the electrode as will be discussed more fully below. The drive tube grip also contains a clamping mechanism (not shown) which selectively allows the electrode wires to pass through the guide tube or to fix these components in a given relative position.

At the distal end of the drive tube 3, two small diametrically opposed and longitudinally extending slots are provided. Extending longitudinally from the upper side of hub 4 is a rectangular, diametrically extending rectangular reference electrode 9 (shown edge-on in the figure). The edges of this electrode are releasably received within the slots in the distal end of the guide tube to form a dog clutch arrangement. Thus, the location of the reference electrode 9 in the slots at the distal end of the guide tube 2 engage the hub 4 with the guide tube 2 such that rotation of the guide tube rotates the hub.

The hub itself is formed of a plastics dielectric material. Extending from its lower end is spiral tip 5. The two electrode wires 8 extend into the upper surface of the hub 4 within which they make electrical connection respectively with the spiral tip 5 and reference electrode 9. The wires are sealed into the hub 4.

The spiral tip 5 has a sharp point at its distal end. It is formed of stainless steel grade 904L which has the characteristic of being essentially non-magnetic and non-magnetisable. Its purpose is to form an electrical connection with the body of the fetus. This is achieved by its pointed tip piercing the scalp and then by twisting the hub so that the spiral tip is drawn into the skin of the fetus. The hub 4 abuts the fetal scalp when the spiral tip 5 has been fully inserted, thereby preventing the electrode from being pushed too deeply under the skin and avoiding any possible injury to the fetus. Furthermore, it electrically isolates the spiral 5 from the amniotic fluid surrounding the fetus. Reference electrode 9, however, is in contact with the amniotic fluid whereby it may provide a second electrical contact.

By connecting the two electrode wires 8 to conventional monitoring apparatus a so-called bipolar connection configuration is provided. However, the electrode is also suitable for, and indeed primarily intended for use in the unipolar configuration in which the spiral tip 5 provides one electrical connection to the fetus with a second electrical connection being provided remotely, for example on the maternal thigh. In the basic form of this configuration the electrode wire connected to the reference electrode is not used at all, and could be removed. However, it may be useful to provide unipolar and bipolar configurations simultaneously for different types of monitoring equipment. For example, the bipolar connection may provide a useful heart rate indication with the spiral electrode and a remote electrode being used to provide S-T wave monitoring.

The electrode is designed to be attached to the presenting part of the fetus, typically the fetal scalp during labour. With the spiral tip 5 withdrawn within the guide tube 2, the distal end of the guide tube 2 is held firmly against the fetal scalp. The drive tube 3 is then advanced through the guide tube 2 until the spiral tip 5 reaches the fetus. The guide tube grip 7 is then used to rotate the guide tube, thereby inserting the spiral tip into the fetal scalp in a corkscrew like manner. Approximately one rotation fully engages the spiral tip leaving the hub 4 pressed against the fetal scalp.

The clamp on the drive tube grip is then released, thereby allowing the drive tube 3 and the drive tube grip 7, together with the guide tube 2, to be withdrawn over the electrode wires 8. The wires may then be attached to approximate monitoring equipment.

After use the electrode is removed by rotating it anti-clockwise.

1. A fetal scalp electrode assembly having a scalp electrode formed of a conductive material wherein the material is of a type which is substantially nonmagnetic and furthermore does not become significantly magnetic after being cold worked.

2. An electrode assembly as claimed in claim 1 wherein the electrode is formed of stainless steel.

3. An electrode assembly as claimed in claim 1 or 2 wherein the relative permeability of the electrode is less than 5.

4. An electrode assembly as claimed in claim 3 wherein the relative permeability of the electrode is less than 2.

5. An electrode assembly as claimed in claim 4 wherein the relative permeability of the electrode is less than 1.2.

6. An electrode assembly as claimed in claim 5 wherein the relative permeability of the electrode is in the range of 1.05-1.2.

7. An electrode assembly as claimed in any preceding claim wherein the electrode is formed of fully austenitic stainless steel.

8. An electrode assembly as claimed in claim 7 wherein the electrode is formed of stainless steel grade 904L (EN 1.4539 or ASTM N08904).

9. An electrode assembly as claimed in any preceding claim wherein the electrode comprises a single spiral arranged for connection to the fetal scalp.

10. An electrode assembly as claimed in claim 9 having no other electrode.

11. An electrode assembly as claimed in any preceding claim in combination with apparatus for studying the S-T interval of an electrode cardiogram signal from a fetus.

12. A method of manufacturing a fetal scalp electrode assembly comprising the steps of selecting a grade of material which is not magnetic even after cold working and forming scalp electrode of the electrode assembly therefrom.

13. A method as claimed in claim 12 wherein the material is stainless steel type 904L.

14. A method of fetal monitoring comprising the use of an electrode assembly as claimed in any of claims 1 to 11.
